(19) United States
(12) Patent Application Publication
(10) Pub. No.: US 2015/0011471 A1
(43) Pub. Date: Jan. 8, 2015

(54) REGENERATION OF ISLET BETA CELLS BY HSP60 DERIVED PEPTIDES

(71) Applicant: Yeda Research and Development Co. Ltd., Rehovot (IL)

(72) Inventors: Irun R. Cohen, Rehovot (IL); Raanan Margalit, Rehovot (IL)

(21) Appl. No.: 14/379,772

(22) PCT Filed: Feb. 28, 2013
(86) PCT No.: PCT/IL2013/050177

§ 371 Date: Aug. 20, 2014

Related U.S. Application Data
(60) Provisional application No. 61/605,274, filed on Mar. 1, 2012.

(51) Int. Cl. A61K 38/17

(52) U.S. Cl. CPC A61K 38/1709
USPC 514/7.3; 530/326

(57) ABSTRACT

The present invention provides compositions for use in the treatment of long-established type 1 diabetes (T1D) using peptides and analogs of heat shock protein 60 (Hsp60). The invention is exemplified using DiaPep277, a peptide analog of human Hsp60, which is shown to induce increase in plasma C-peptide and regeneration of islet beta cells. The invention further relates to regimens useful for treatment of long established T1D in patients having no demonstrable islet beta cell-function.

### FIELD OF THE INVENTION

The invention relates to methods for regeneration of pancreatic islet beta cells and treatment of type 1 diabetes (T1D) in patients having long-standing disease and no residual beta cell function. The method comprises administration of a peptide derived from heat shock protein 60 (Hsp60), or an analog thereof e.g. DiaPep277.

### BACKGROUND OF THE INVENTION

Type 1 diabetes (T1D, also referred to as insulin dependent diabetes mellitus, IDDM) is an autoimmune disease that results in the destruction of the beta-cells in the pancreas. The collapse of glucose homeostasis and clinical manifestation of the disease is thought to occur only after 80-90% of pancreatic beta cells have been inactivated by the immune response. Incipient diabetes can be diagnosed by the detection of immunological markers of beta-cell autoimmunity only after the onset of the autoimmune process.

C-peptide is a protein fragment cleaved from pro-insulin by beta cells during the endogenous biosynthesis of insulin by these cells. C-peptide is released into the circulation from the secretory granules in a 1:1 molar ratio with insulin. Plasma C-peptide provides a surrogate measure of endogenous insulin production and reflects the activity of beta cells. It was recently suggested (David J Klinke PLoS ONE 2011, 6, e26873) that plasma C-peptide may provide a surrogate measure of beta-cell mass in humans.

Various self-antigens targeted by an autoimmune process have been suggested to play a role in the development of diabetes. Indeed, antibodies against glutamic acid decarboxylase (GAD), insulin, islet cell antigen (ICA-69), and Hsp60 have been found in the circulation at the onset of diabetes in humans (Cohen IR 2002, Diabetologia 45,1468-1474), and in pre-diabetic non-obese diabetic (NOD)-mice (Brudzynski, 1993, Diabetes 42, 908-13.) and biobreeding (BB) rats.

Heat shock proteins (HSPs) are highly conserved proteins expressed in all prokaryotic and eukaryotic cells. They are involved in many important cellular processes such as correct folding of newly synthesized proteins and subunit assembly and therefore are termed molecular chaperones (Bukau, B., et al. 2000, Cell 101, 119-122). Under non-physiological conditions like high temperature, ultraviolet radiation, viral or bacterial infection, cellular HSP synthesis is up-regulated. HSPs exert cytoprotective functions such as preventing the aggregation of denatured proteins, initiating their refolding or proteolytic degradation. According to their molecular weight, HSPs are divided into six subfamilies: small HSPs, HSP40, HSP60, HSP70, HSP90 and HSP100. They are located in the cytosol (HSP70, HSP90, HSP100), in the endoplasmic reticulum (HSP70, HSP90) or in mitochondria (HSP60) of cells in general and in the cytoplasm and on the surface of beta cells during the diabetogenic process (Brudzynski, 1993, ibid).

The HSP60, HSP70, and HSP90 subfamilies have attracted increasing attention because of their potential roles in immunologically relevant processes. Several studies have identified HSPs as targets of immune responses during microbial infections (Zugel, U., and Kaufmann, S. H., 1999, Immunobiology 201, 22-35). Because of the high sequence homology between microbial HSPs and endogenous HSPs derived from damaged or stressed tissue, immunological cross-reactivity was suggested to contribute to the development of autoimmune disorders including rheumatoid arthritis and diabetes (Holoshitz, J., et al. 1986, Lancet 2, 305-309; Elias, D., et al., 1991, Proc. Natl. Acad. Sci. U.S.A. 88, 3088-3091; Abulafia-Lapid, R., et al., 1999, J. Autoimmun. 12, 121-129).

Many disclosures claim uses of heat shock proteins or fragments thereof as immune modulators in diagnosis, treatment or prevention of autoimmune diseases. Most of these disclosures relate to Hsp60, or fragments of this protein. Antibodies against human Hsp60, which have a high homology to bacterial Hsp65, have been found in the circulation at the onset of T1D in humans and in pre-diabetic NOD-mice. Elias et al. (Diabetes 1997, 46, 758-64) demonstrated a specific peptide of human Hsp60, denoted p277, to be one of the immunodominant epitopes in autoimmune diabetes. Accordingly, T-cell reactivity to p277 has been reported at the onset of diabetes in NOD mice. Subcutaneous administration of p277 down-regulated T-cell reactivity to beta cell antigens and prevented the development of diabetes in NOD mice. Treatment induced p277-specific IgG1 antibodies as well as an increase in p277-specific IL-4 and IL-10 secretion and a decrease in gamma interferon secretion, suggesting an up-regulation of the Th2 cytokine pathway. As destruction of the islets of Langerhans in the pancreas is believed to be a Th1 response, a shift of Th1 to Th2 response caused by p277 may be involved in the attenuation of T1D.

U.S. Pat. Nos. 5,114,844; 5,671,848; 5,578,303 and 5,780,034 disclose the use of Hsp60 in diagnosis and treatment of T1D. It has been further disclosed (U.S. Pat. Nos. 6,180,103 and 5,993,803 and WO 96/19236, WO 97/01959 and WO 98/08536) that fragments and peptide analogs of this Hsp60 protein may serve as therapeutically useful entities in preventing or alleviating T1D and host vs. graft disease.

A peptide analog of human Hsp60 p277, denoted DiaPep277, disclosed in U.S. Pat. No. 6,180,103 and WO 96/19236 as p277(Val⁶,Val¹¹) is a synthetic analog in which the two native cysteine residues at positions 6 and 11 were replaced with Valine residues.

WO 03/070761 discloses anti-inflammatory Hsp60 derived peptides including a minimal epitope of the peptide p277 that are capable of reacting via the Toll like receptor 2 (Tlr2) on T cells.

WO 2005/072056 discloses the use of DiaPep277, in conjunction with low-antigenicity diet, for delaying the onset of autoimmune diseases, particularly Type 1 diabetes, and to methods useful for prevention, delay, suppression or treatment of autoimmune diseases using oral administration of DiaPep277.

WO 2006/072946 discloses the use of p277 and its analogs in modulation of immune responses and inflammatory diseases and specifically in the treatment or prevention of hepatic disorders.

U.S. Pat. No. 8,211,430 discloses combination therapies for T1D comprising an agent, that stimulates pancreatic islet cell regeneration, including specifically human proIslet Peptides (HIP), together with an agent that inhibits the activity of or blocks or destroys the autoimmune cells that target pancreatic islet cells, such as inter alia heat shock proteins.

DiaPep277 has successfully completed a phase III clinical trial for the treatment of subjects with new onset T1D. It preserves the residual beta cells in T1D by modulating and arresting the autoimmune destruction of beta cells. DiaPep277 has not been previously thought capable of treating T1D subjects who have no demonstrable residual beta cells function as determined by fasting C-peptide levels. Nowhere in the prior art is it taught or suggested that the immunomodulatory peptides derived from Hsp60 are capable of inducing pancreatic cell regeneration.

There is an unmet need to provide effective compositions for treatment of long-established T1D, and to elicit regeneration of beta cells.

### SUMMARY OF THE INVENTION

The present invention provides pharmaceutical compositions comprising the Hsp60 peptide analog DiaPep277 (SEQ ID NO: 2) or other Hsp60 derived peptides and peptide analogs, useful in methods of stimulating beta cell regenerating in subjects having no demonstrable residual beta cells or beta-cell function. The present invention provides the use of heat shock protein 60 (Hsp60)-derived peptides and their analogs, in particular the peptide denoted DiaPep277, for treatment of long-established T1D in patients having no demonstrable islet beta cells or beta-cell function. It was unexpectedly found that DiaPep277 which was shown to be effective in treatment of newly-diagnosed T1D by modulating and arresting the autoimmune destruction of residual beta cells is useful also in long-established disease, by stimulating regeneration of new beta cells in the pancreatic islets. It was further found that schedules used for treating newly diagnosed T1D, having residual functional beta cells, with DiaPep277 are ineffective in long established disease and therefore new treatment schedules and formulations are herein provided. It was surprisingly found that specific and more frequent doses of DiaPep277 are effective in inducing regeneration and decreasing lymphocyte infiltrates to beta cell islets. According to the present invention, a novel patient population of T1D subjects is provided as a target for treatment with DiaPep277, consisting of subjects having long-established disease and no demonstrable residual beta cells, as measured by C-peptide levels.

It is now disclosed for the first time that Hsp60 derived peptides previously known to be immuno-modulatory are unexpectedly capable of inducing beta cell regeneration without administration of any additional exogenous agent known to stimulate beta islet production. It is also disclosed for the first time that the stimulation of beta islet cells necessitates sustained or frequent exposure to the Hsp60 derived peptides in order to achieve the required activity. The dosages of DiaPep277 utilized heretofore both clinically and preclinically to achieve a Th1 to Th2 shift do not reach the threshold required to elicit the beta cell stimulatory effect.

According to one aspect the invention provides a method of inducing regeneration of beta cell islets in a patient having T1D, the method consisting of administering a peptide derived from Hsp60 or a peptide analog thereof to thereby increasing the beta cell mass in a patient having T1D.

According to another aspect the invention provides a method of inducing regeneration of beta cell islets in a patient having T1D, the method comprising administering a pharmaceutical composition comprising a peptide derived from Hsp60 or a peptide analog thereof as the sole active ingredient to stimulate beta cell regeneration thereby increasing the beta cell mass in a patient having T1D.

According to specific embodiments the patient has long established T1D; according to specific embodiments the patient has a fasting level of no more than 0.2 nM of C peptide; according to specific embodiments the patient has diagnosed T1D for more than one year.

According to some embodiments, the peptide analog consists a sequence corresponding to amino acid residues 437-460 of human Hsp60 having the sequence: Val-Leu-Gly-Gly-Gly-X₁-Ala-Leu-Leu-Arg-X₂-Ile-Pro-Ala-Leu-Asp-Ser-Leu-X₃-Pro-Ala-Asn-Glu-Asp (SEQ ID NO:1), wherein X1 is a Cys or Val residue, X₂ is a Cys or Val residue, and X₃ is a Thr or Lys residue.

According to particular embodiments, X₁ and X₂ are Val and X₃ is Thr and the peptide analog is 24-30 amino acids long.

According to a particular embodiment, the peptide analog is a Val⁶, Val¹¹ analog of residues 437-460 of Hsp60, as set forth in SEQ ID NO: 2:

(SEQ ID NO: 2)
1 6 11
Val-Leu-Gly-Gly-Gly-Val-Ala-Leu-Leu-Arg-Val-
Ile-Pro-Ala-Leu-Asp-Ser-Leu-Thr-Pro-
24
Ala-Asn-Glu-Asp, herein denoted DiaPep277.

According to another embodiment, the Hsp60 fragment peptide is selected from the group consisting of:

residues 31-50 of human Hsp60:
(SEQ ID NO: 3)
Lys-Phe-Gly-Ala-Asp-Ala-Arg-Ala-Leu-Met-Leu-
Gln-Gly-Val-Asp-Leu-Leu-Ala-Asp-Ala;
residues 136-155 of human Hsp60:
(SEQ ID NO: 4)
Asn-Pro-Val-Glu-Ile-Arg-Arg-Gly-Val-Met-Leu-
Ala-Val-Asp-Ala-Val-Ile-Ala-Glu-Leu;
residues 151-170 of human Hsp60:
(SEQ ID NO: 5)
Val-Ile-Ala-Glu-Leu-Lys-Lys-Gln-Ser-Lys-Pro-
Val-Thr-Thr-Pro-Glu-Glu-Ile-Ala-Gln;
residues 166-185 of human Hsp60:
(SEQ ID NO: 6)
Glu-Glu-Ile-Ala-Gln-Val-Ala-Thr-Ile-Ser-Ala-
Asn-Gly-Asp-Lys-Glu-Ile-Gly-Asn-Ile;
residues 195-214 of human Hsp60:
(SEQ ID NO: 7)
Arg-Lys-Gly-Val-Ile-Thr-Val-Lys-Asp-Gly-Lys-
Thr-Leu-Asn-Asp-Glu-Leu-Glu-Ile-Ile;
residues 255-274 of human Hsp60:
(SEQ ID NO: 8)
Gln-Ser-Ile-Val-Pro-Ala-Leu-Glu-Ile-Ala-Asn-
Ala-His-Arg-Lys-Pro-Leu-Val-Ile-Ile;
residues 286-305 of human Hsp60:
(SEQ ID NO: 9)
Leu-Val-Leu-Asn-Arg-Leu-Lys-Val-Gly-Leu-Gln-
Val-Val-Ala-Val-Lys-Ala-Pro-Gly-Phe;
residues 346-365 of human Hsp60:
(SEQ ID NO: 10)
Gly-Glu-Val-Ile-Val-Thr-Lys-Asp-Asp-Ala-Met-
Leu-Leu-Lys-Gly-Lys-Gly-Asp-Lys-Ala;
residues 421-440 of human Hsp60:
(SEQ ID NO: 11)
Val-Thr-Asp-Ala-Leu-Asn-Ala-Thr-Arg-Ala-Ala-
Val-Glu-Glu-Gly-Ile-Val-Leu-Gly-Gly;
residues 436-455 of human Hsp60:
(SEQ ID NO: 12)
Ile-Val-Leu-Gly-Gly-Gly-Cys-Ala-Leu-Leu-Arg-
Cys-Ile-Pro-Ala-Leu-Asp-Ser-Leu-Thr;
residues 466-485 of human Hsp60:
(SEQ ID NO: 13)
Glu-Ile-Ile-Lys-Arg-Thr-Leu-Lys-Ile-Pro-Ala-
Met-Thr-Ile-Ala-Lys-Asn-Ala-Gly-Val;
residues 511-530 of human Hsp60:
(SEQ ID NO: 14)
Val-Asn-Met-Val-Glu-Lys-Gly-Ile-Ile-Asp-Pro-
Thr-Lys-Val-Val-Arg-Thr-Ala-Leu-Leu;
residues 343-366 of human Hsp60:
(SEQ ID NO: 15)
Gly-Lys-Val-Gly-Glu-Val-Ile-Val-Thr-Lys-Asp-
Asp-Ala-Met.

According to some embodiments at dose of at least 2 mg of the Hsp60 derived peptide or peptide analog is administered at least once monthly. According to other embodiments, the at least once monthly dose is of at least 5 mg. According to yet other embodiments, the Hsp60 derived peptide or peptide analog is administered at least 4 times per month. According to some embodiments, a dose of at least 2 mg of Hsp60 derived peptide or peptide analog is administered 1-4 times per week. Each possibility represents a separate embodiment of the present invention.

According to particular embodiments, the method comprises administering of a long acting pharmaceutical composition comprising a therapeutically effective amount of Hsp60 derived peptide or peptide analog in a depot form.

The present invention provides, according to another aspect, a method of inducing or stimulating regeneration of beta cells, comprising applying a peptide derived from Hsp60 or a peptide analog thereof to the beta cells.

According to some embodiments, the method of inducing or stimulating regeneration of beta cells comprises administration of a peptide derived from Hsp60 or a peptide analog thereof to a subject in need thereof.

According to yet other embodiments, a culture or explant comprising stem cells is exposed to a peptide derived from Hsp60 or to a peptide analog thereof.

According to another aspect the invention provides a method of transformation from pancreatic ductal cells into islet cells comprising administering to a subject in need thereof a peptide derived from Hsp60 or a peptide analog thereof.

According to some embodiments the peptide analog is DiaPep277 (SEQ ID NO: 2).

According to another aspect the present invention provides a method of increasing beta cell mass, comprising administering to a subject in need thereof a peptide derived from Hsp60 or a peptide analog thereof.

According to some embodiments the peptide analog is DiaPep277 (SEQ ID NO: 2).

According to some embodiments, the patient achieves restoration of normal glucose metabolism.

According to other embodiments, the patient achieves restoration of normal hormonal function.

It is to be explicitly understood that methods comprising co-administration of human proIslet Peptides (HIP), or other agents that stimulate pancreatic islet cell regeneration together with a peptide derived from Hsp60 or a peptide analog thereof, are excluded from the scope of the present invention.

It will be understood to one of skill in the art that the methods and compositions of the invention can also be employed as adjunctive therapy to insulin therapy in T1D. Specifically, the methods will be useful in patients with long established diabetes, in children and adults to improve normoglycemia, and in patients with poorly controlled diabetes, and recurrent hypoglycemia in T1D.

The present invention provides, according to another aspect, a method of treating of long established T1D comprising administering to a subject in need thereof, a composition comprising a peptide derived from Hsp60 or a peptide analog thereof.

According to the present invention, a patient suffering from long established T1D has a fasting level of C-peptide of 0.2 nM or less or has clinically established T1D for a period of a year or more.

According to some embodiments, the peptide analog consists a sequence corresponding to amino acid residues 437-460 of human Hsp60 having the sequence: Val-Leu-Gly-Gly-Gly-X₁-Ala-Leu-Leu-Arg-X₂-Ile-Pro-Ala-Leu-Asp- Ser-Leu-X₃-Pro-Ala-Asn-Glu-Asp (SEQ ID NO:1), wherein X1 is a Cys or Val residue, X₂ is a Cys or Val residue, and X₃ is a Thr or Lys residue.

According to particular embodiments, X₁ and X₂ are Val and X₃ is Thr and the peptide analog is 24-30 amino acids long.

According to a particular embodiment, the peptide analog is a Val⁶, Val¹¹ analog of residues 437-460 of Hsp60, as set forth in SEQ ID NO: 2:

(SEQ ID NO: 2)
1 6 11
Val-Leu-Gly-Gly-Gly-Val-Ala-Leu-Leu-Arg-Val-
Ile-Pro-Ala-Leu-Asp-Ser-Leu-Thr-Pro-
24
Ala-Asn-Glu-Asp, herein denoted DiaPep277.

According to another embodiment, the Hsp60 fragment peptide is selected from the group consisting of:

residues 31-50 of human Hsp60:
(SEQ ID NO: 3)
Lys-Phe-Gly-Ala-Asp-Ala-Arg-Ala-Leu-Met-Leu-Gln-
Gly-Val-Asp-Leu-Leu-Ala-Asp-Ala;
residues 136-155 of human Hsp60:
(SEQ ID NO: 4)
Asn-Pro-Val-Glu-Ile-Arg-Arg-Gly-Val-Met-Leu-Ala-
Val-Asp-Ala-Val-Ile-Ala-Glu-Leu;
residues 151-170 of human Hsp60:
(SEQ ID NO: 5)
Val-Ile-Ala-Glu-Leu-Lys-Lys-Gln-Ser-Lys-Pro-Val-
Thr-Thr-Pro-Glu-Glu-Ile-Ala-Gln;
residues 166-185 of human Hsp60:
(SEQ ID NO: 6)
Glu-Glu-Ile-Ala-Gln-Val-Ala-Thr-Ile-Ser-Ala-Asn-
Gly-Asp-Lys-Glu-Ile-Gly-Asn-Ile;
residues 195-214 of human Hsp60:
(SEQ ID NO: 7)
Arg-Lys-Gly-Val-Ile-Thr-Val-Lys-Asp-Gly-Lys-Thr-
Leu-Asn-Asp-Glu-Leu-Glu-Ile-Ile;
residues 255-274 of human Hsp60:
(SEQ ID NO: 8)
Gln-Ser-Ile-Val-Pro-Ala-Leu-Glu-Ile-Ala-Asn-Ala-
His-Arg-Lys-Pro-Leu-Val-Ile-Ile;
residues 286-305 of human Hsp60:
(SEQ ID NO: 9)
Leu-Val-Leu-Asn-Arg-Leu-Lys-Val-Gly-Leu-Gln-Val-
Val-Ala-Val-Lys-Ala-Pro-Gly-Phe;
residues 346-365 of human Hsp60:
(SEQ ID NO: 10)
Gly-Glu-Val-Ile-Val-Thr-Lys-Asp-Asp-Ala-Met-Leu-
Leu-Lys-Gly-Lys-Gly-Asp-Lys-Ala;
residues 421-440 of human Hsp60:
(SEQ ID NO: 11)
Val-Thr-Asp-Ala-Leu-Asn-Ala-Thr-Arg-Ala-Ala-Val-
Glu-Glu-Gly-Ile-Val-Leu-Gly-Gly;
residues 436-455 of human Hsp60:
(SEQ ID NO: 12)
Ile-Val-Leu-Gly-Gly-Gly-Cys-Ala-Leu-Leu-Arg-Cys-
Ile-Pro-Ala-Leu-Asp-Ser-Leu-Thr;
residues 466-485 of human Hsp60:
(SEQ ID NO: 13)
Glu-Ile-Ile-Lys-Arg-Thr-Leu-Lys-Ile-Pro-Ala-Met-
Thr-Ile-Ala-Lys-Asn-Ala-Gly-Val;
residues 511-530 of human Hsp60:
(SEQ ID NO: 14)
Val-Asn-Met-Val-Glu-Lys-Gly-Ile-Ile-Asp-Pro-Thr-
Lys-Val-Val-Arg-Thr-Ala-Leu-Leu;
residues 343-366 of human Hsp60:
(SEQ ID NO: 15)
Gly-Lys-Val-Gly-Glu-Val-Ile-Val-Thr-Lys-Asp-Asp-
Ala-Met.

The Hsp60 peptide or analog is administered, according to the invention, within a pharmaceutical composition comprising at least one pharmaceutically acceptable excipient, diluent, adjuvant or salt. The pharmaceutical composition may be administered to a subject in need thereof, by any administration route, including but not limited to: intramuscular, intravenous, oral, intraperitoneal, subcutaneous, topical, intradermal or transdermal delivery.

According to some embodiments, the composition is administered by a parenteral route selected from the group consisting of: subcutaneous injection (SC), intra-peritoneal (IP) injection, intra-muscular (IM) injection and intra-venous (IV) injection. According to a particular embodiment, the composition is administered orally (PO). According to some embodiments the compositions useful in the methods of the present invention are devoid of adjuvants.

According to alternative embodiments, the composition comprises an adjuvant or other agent that may stimulate the response to Hsp derived peptide or analog. Pharmaceutically acceptable adjuvants include, but are not limited to oil in water emulsions, microemulsions and liposomes.

According to yet other embodiment the pharmaceutical composition comprises Hsp60 derived peptide or peptide analog in an aqueous solution, including but not limited to saline, PBS, water.

In some embodiments the composition is formulated for intramuscular, intravenous, oral, intraperitoneal, subcutaneous, topical, intradermal or transdermal delivery.

According to some embodiments, the composition comprises an individual dose of at least 2 mg of the Hsp60 derived peptide or peptide analog. According to other embodiments, the individual dose is of at least 5 mg of the Hsp60 derived peptide or peptide analog. According to specific embodiments, the individual dose is of 10 mg of the Hsp60 derived peptide or peptide analog. Each possibility represents a separate embodiment of the present invention.

According to some embodiments, the Hsp60 derived peptide or peptide analog is administered to a subject in need thereof at least once a month, for at least one year. According to other embodiments, the Hsp60 derived peptide or peptide analog is administered to a subject in need thereof at least once a week, for at least one year. According to specific embodiments, the Hsp60 derived peptide or peptide analog is administered to a subject in need thereof in multiples doses per week. According to some embodiments, the Hsp60 derived peptide or peptide analog is administered to the subject 2, 3, 4, 5, 6 or 7 times per week. Each possibility represents a separate embodiment of the present invention.

According to some particular embodiments, a composition comprising 2-50 mg of the Hsp60 derived peptide or peptide analog is administered to a subject in need thereof 1-20 times per month by a route selected from the group consisting of: subcutaneous injection, intra-peritoneal (IP) injection, intra-muscular (IM) injection and intra-venous (IV) injection. According to a particular embodiment, the pharmaceutical composition administered by subcutaneous injection comprises an oil in water emulsion. Each possibility represents a separate embodiment of the present invention. According to other particular embodiments, a composition comprising 2-10 mg of the Hsp60 derived peptide or peptide analog is administered to a subject in need thereof 1-4 times per week. Each possibility represents a separate embodiment of the present invention.

According to some specific embodiments a dose of 5 mg DiaPep277 is administered SC at least once a week to a subject suffering from long established T1D. According to other embodiments, a composition comprising 50-500 mg Hsp60 peptide or analog is provided for oral administration to a subject in need thereof following a schedule of at least once a month for at least a year. According to some specific embodiments, a dose of about 100 mg of Hsp60 peptide analog in aqueous solution is administered to a subject in need thereof, at least once a week, for at least one year. Each possibility represents a separate embodiment of the present invention.

According to some embodiments and Hsp60 peptide analog is DiaPep277 (SEQ ID NO: 2).

The present invention also discloses Hsp60 derived peptides and analogs for use in treating T1D in a subpopulation of patients having no demonstrable functional pancreatic beta cells, as measured by plasma C-peptide levels.

Also disclosed is the use of Hsp60 derived peptide or analog for preparation of a medicament for treatment of T1D patients having no demonstrable pancreatic beta cells, as measured by plasma C-peptide levels.

Also provided according to the present invention are pharmaceutical compositions comprising Hsp60 derived peptide or analog for inducing beta cell regeneration in T1D patients and pharmaceutical compositions comprising Hsp60 derived peptide or analog as the sole active ingredient, for treatment of patients having long-established T1D as determined by C-peptide level.

According to particular embodiments, the Hsp60 peptide analog is DiaPep277 (SEQ ID NO: 2).

Use of an Hsp60 peptide or analog, or a pharmaceutical composition comprising it, for inducing regeneration of beta cells is also within the scope of the present invention.

According to particular embodiments, the Hsp60 peptide analog is DiaPep277 (SEQ ID NO: 2).

The present invention provides according to another aspect long acting pharmaceutical compositions comprising DiaPep277 or a pharmaceutically acceptable salt thereof, specifically formulated for providing a therapeutically effective amount of the peptide over a period selected from 2-6 days, one week, two weeks or longer.

According to some embodiments, the long acting pharmaceutical composition is for use in inducing regeneration of beta cell islets in a patient having T1D.

According to other embodiments, the long acting pharmaceutical composition is for use in treatment of long established T1D in subjects having a fasting C-peptide level of 0.2 nM or less or having a clinically established T1D for a period of a year or more.

According to some specific embodiments, the long acting pharmaceutical composition is provided in depot form suitable for injection or implantation at a medically acceptable location in a subject in need thereof.

According to some embodiments, the long acting pharmaceutical composition is suitable for a dosing schedule from once weekly to once in every 6 months.

According to particular embodiments, the composition is suitable for a dosing schedule from once every 2 weeks to once monthly. Each possibility represents a separate embodiment of the present invention.

Specific examples of the long acting compositions include biodegradable or non-biodegradable microspheres, implants of any suitable geometric shape, implantable rods, implantable capsules, implantable rings, prolonged release gels and erodible matrices. Each possibility represents a separate embodiment of the invention.

The present invention further provides a method of treating long-established T1D, comprising the administration or implantation of a composition comprising a therapeutically effective amount of a pharmaceutically acceptable salt of DiaPep277.

The long acting pharmaceutical compositions according to the principles of the present invention provide equal or superior therapeutic efficacy to the commercially available injectable dosage forms, with reduced incidence and/or severity of side effects at the local and/or systemic levels.

According to certain embodiments, an implantable depot comprising DiaPep277 suitable for subcutaneous or intramuscular implantation is provided.

According to alternative embodiments, the long acting parenteral pharmaceutical composition comprises a pharmaceutically acceptable biodegradable or non-biodegradable carrier for DiaPep277.

According to some embodiments, the carrier is selected from PLGA, PLA, PGA, polycaprolactone, polyhydroxybutyrate, polyorthoesters, polyalkaneanhydrides, gelatin, collagen, oxidized cellulose, and polyphosphazene. Each possibility represents a separate embodiment of the invention.

According to particular embodiments, the long acting pharmaceutical compositions of the present invention are in the form of microparticles prepared by a water-in oil-in water double emulsification process. In currently preferred embodiments, the long acting pharmaceutical compositions of the present invention comprise an internal aqueous phase comprising a therapeutically effective amount of a pharmaceutically acceptable salt of DiaPep277, a water immiscible polymeric phase comprising a carrier selected from a biodegradable and a non-biodegradable polymer, and an external aqueous phase. In other currently preferred embodiments, the water immiscible polymeric phase comprises a biodegradable polymer selected from PLA and PLGA. Each possibility represents a separate embodiment of the invention. In additional embodiments, the external aqueous phase comprises a surfactant selected from polyvinyl alcohol (PVA), polysorbate, polyethylene oxide-polypropylene oxide block copolymers and cellulose esters. Each possibility represents a separate embodiment of the invention.

According to another aspect, the present invention provides a kit comprising DiaPep277 in a dose form of at least 2 mg formulated for administration to patients having T1D. According to other embodiments, the kit comprises 5 mg of DiaPe277. The kit may further comprise instructions for the administration of the composition.

### BRIEF DESCRIPTION OF THE DRAWINGS

FIG. 1 is a photograph of a histological section of pancreas taken from a control NOD mouse which received vehicle only. Lymphocyte infiltrates are clearly visible.

FIG. 2 is a photography of a histological section of pancreas taken from an experimental NOD mouse treated SC with 100 μg of DiaPep277 once a week. Lymphocyte infiltrates are not seen in this newly developed islet.

FIG. 3 is a photograph of a histological section of pancreas taken from an experimental NOD mouse treated SC with 500 μg, once a week. Beta cells in the islet are clear from lymphocyte infiltrates.

### DETAILED DESCRIPTION OF THE INVENTION

Hsp60 derived peptides and peptide analogs, especially the peptide analog DiaPep277, are known to be effective in treatment of T1D by modulating and arresting the autoimmune destruction of residual beta cells. Such treatment was known to be effective in the early stages of the disease in which beta cells still exist within the patient's pancreatic islets. To exhibit symptoms of T1D, approximately 80-90 percent of the pancreatic islets are either destroyed or non-functional. Therefore, 20 percent may still be remaining. This stage is sometimes known as “the honeymoon period”. Patients who experience a honeymoon period notice that they experience normoglycemia (normal blood sugars) with relatively low doses of insulin due to some of their own endogenous insulin working additively with the injected dose of insulin. Over time, patients require more exogenous insulin as their residual beta cells lose function, until the complete inactivity of the beta cells. It was now unexpectedly found that certain HspHsp60 derived peptides and peptide analogs are also effective in inducing a return of endogenous insulin production detected by rising levels of C-peptide as a sign of regeneration of beta cells in subjects with long-established disease (over a year after onset). These subjects have and no demonstrable islet beta cells before treatment (as demonstrated by little or no plasma C-peptide levels). For treatment of such subjects, dose and schedules which are known to be effective in treatment of newly-diagnosed T1D patients cannot be used, since the subjects have no residual beta cells. Alternative doses and treatment schedules are thus provided.

The effective treatment of newly-diagnosed T1D with DiaPep277 is 1 mg administered every three months by subcutaneous (SC) injection. According to the present invention an exemplary dose of DiaPep277 for long-established T1D is 2 mg or greater administered by SC injection, by intra-peritoneal (IP) injection, by intra-venous (IV) injection, by intra-muscular (IM) injection or by oral administration (PO). Typically, for subcutaneous injection DiaPep277 is formulated with an adjuvant, such as an oil in water emulsion while for the IP, oral and IV routes of injection the peptide are formulated according to some embodiments in aqueous solutions. Other formulations and schedules are possible according to the present invention. DiaPep277, typically without an adjuvant, may be also administered orally at doses of 100 mg or greater. The frequency of administration is, according to some embodiments of the present invention, once or twice a month for at least a year, weekly in the case of oral administration.

Pharmaceutical compositions comprising HspHsp60 derived peptides and analogs are also disclosed in the present invention, together with novel formulations and treatment schedules, for use in treatment of established, long-established T1D in patients having no residual beta cells.

Without wishing to be limited to any theory, it is suggested that DiaPep277 and other HspHsp60 derived peptides and analogs, facilitate the re-generation of beta cells by activating stem cells residing in the pancreatic islets to differentiate into beta cells in the target tissue.

The new methods provided by the present invention reverse the underlying pathologic mechanisms of diseases and conditions resulting from decreased insulin production due to an imbalance between destruction, and regeneration of insulin producing islet cells. The methods and compounds of the invention can reduce the insulin requirements of patients currently taking insulin due to having T1D and can improve glucose control in such patients.

As disclosed herein, in vivo animal models are used to assess the effectiveness of the HspHsp60 peptides and peptide analogs in long-established T1D to effect induction of generation of beta cells in mice. In some of these models, NOD mice that spontaneously develop T1D are used and induced to develop increased C-peptide in advanced disease. Disease progression and effectiveness of treatment are measured by determining glucose tolerance, fasting glucose and plasma C-peptide levels as known in the art.

### Terminology and Definitions:

“Functional derivatives” of the peptides of the invention as used herein covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide, do not confer toxic properties on compositions containing it and do not adversely affect the antigenic properties thereof.

These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “analog” further indicates a molecule which has the amino acid sequence according to the invention except for one or more amino acid changes. Analogs according to the present invention may comprise also peptidomimetics. “Peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The design of appropriate “analogs” may be computer assisted.

An “effective peptide” will have the activity to achieve a desired result, such as induction of increased C-peptide levels. Thus reference to a particular peptide or “analog” includes the naturally occurring peptide sequence or a peptide that has the substantially the same activity as the naturally occurring sequence. “Effective peptides” of the invention also include modified peptides (with amino acid substitutions, both conservative and non-conservative) that have the same activity as a wild-type or unmodified peptide. “Salts” of the peptides of the invention contemplated by the invention are physiologically acceptable organic and inorganic salts.

As used herein and in the claims, the phrase “therapeutically effective amount” means that amount of peptide or peptide analog or composition comprising same to administer to a host to achieve the desired results for the indications disclosed herein.

The amino acids used in this invention are those which are available commercially or are available by routine synthetic methods. Certain residues may require special methods for incorporation into the peptide, and either sequential, divergent and convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

Conservative substitution of amino acids as known to those skilled in the art are within the scope of the present invention. Conservative amino acid substitutions include replacement of one amino acid with another having the same type of functional group or side chain e.g. aliphatic, aromatic, positively charged, negatively charged. These substitutions may enhance oral bioavailability, penetration into the islets, targeting to specific beta cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

### Pharmacology

Apart from other considerations, the fact that the novel active ingredients of the invention are peptides, peptide analogs or peptidomimetics, dictates that the formulation be suitable for delivery of these types of compounds. In general, peptides are less suitable for oral administration due to susceptibility to digestion by gastric acids or intestinal enzymes, but it is now disclosed that the compositions according to the present invention my be also suitable for oral administration. Other routes of administration according to the present invention are intravenous, intramuscular, subcutaneous, or intradermal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping, lyophilizing or liposome capturing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

Pharmaceutical compositions suitable for use in context of the present invention include compositions wherein the active ingredients are contained in an amount effective to achieve the intended purpose. More specifically, a therapeutically effective amount means an amount of a compound effective to increase the C-peptide and endogenous insulin production of the subject being treated. Determination of a therapeutically effective amount is well within the capability of those skilled in the art.

Toxicity and therapeutic efficacy of the fragments and analogs described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the manner of administration, the judgment of the prescribing physician, and all other relevant factors.

In one particularly preferred embodiment according to the present invention, the peptides are administered orally (e.g. as a syrup, capsule, or tablet).

In certain embodiments, peptide delivery can be enhanced by the use of protective excipients. This is typically accomplished either by complexing the peptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy, 1998, Biotechnol. Prog. 14, 108; Johnson et al., 1996, Nature Med. 2, 795; Herbert et al., 1998, Pharmaceut. Res. 15, 357) a dry powder composed of biodegradable polymeric microspheres containing the protein in a polymer matrix that can be compounded as a dry formulation with or without other agents.

In certain embodiments, dosage forms of the compositions of the present invention include, but are not limited to, biodegradable injectable depot systems such as, PLGA based injectable depot systems; non-PLGA based injectable depot systems, and injectable biodegradable gels or dispersions. Each possibility represents a separate embodiment of the invention. The term “biodegradable” as used herein refers to a component which erodes or degrades at its surfaces over time due, at least in part, to contact with substances found in the surrounding tissue fluids, or by cellular action. In particular, the biodegradable component is a polymer such as, but not limited to, lactic acid-based polymers such as polylactides e.g. poly(D,L-lactide) i.e. PLA; glycolic acid-based polymers such as polyglycolides (PGA) e.g. Lactel® from Durect; poly(D,L-lactide-co-glycolide) i.e. PLGA, (Resomer® RG-504, Resomer® RG-502, Resomer® RG-504H, Resomer® RG-502H, Resomer® RG-5045, Resomer® RG-5025, from Boehringer, Lactel® from Durect); polycaprolactones such as Poly(e-caprolactone) i.e. PCL (Lactel® from Durect); polyanhydrides; poly(sebacic acid) SA; poly(ricenolic acid) RA; poly(fumaric acid), FA; poly(fatty acid dimmer), FAD; poly(terephthalic acid), TA; poly(isophthalic acid), IPA; poly(p-{carboxyphenoxy}methane), CPM; poly(p-{carboxyphenoxy}propane), CPP; poly(p-{carboxyphenoxy}hexane)s CPH; polyamines, polyurethanes, polyesteramides, polyorthoesters {CHDM: cis/trans-cyclohexyl dimethanol, HD:1,6-hexanediol. DETOU: (3,9-diethylidene-2,4,8,10-tetraoxaspiro undecane)}; polydioxanones; polyhydroxybutyrates; polyalkylene oxalates; polyamides; polyesteramides; polyurethanes; polyacetals; polyketals; polycarbonates; polyorthocarbonates; polysiloxanes; polyphosphazenes; succinates; hyaluronic acid; poly(malic acid); poly(amino acids); polyhydroxyvalerates; polyalkylene succinates; polyvinylpyrrolidone; polystyrene; synthetic cellulose esters; polyacrylic acids; polybutyric acid; triblock copolymers (PLGA-PEG-PLGA), triblock copolymers (PEG-PLGA-PEG), poly(N-isopropylacrylamide) (PNIPAAm), poly(ethylene oxide)-poly(propylene oxide)-poly(ethylene oxide)tri-block copolymers (PEO-PPO-PEO), poly valeric acid; polyethylene glycol; polyhydroxyalkylcellulose; chitin; chitosan; polyorthoesters and copolymers, terpolymers; lipids such as cholesterol, lecithin; poly(glutamic acid-co-ethyl glutamate) and the like, or mixtures thereof.

In some embodiments, the compositions of the present invention comprise a biodegradable polymer selected from, but not limited to, PLGA, PLA, PGA, polycaprolactone, polyhydroxybutyrate, polyorthoesters, polyalkaneanhydrides, gelatin, collagen, oxidized cellulose, polyphosphazene and the like. Each possibility represents a separate embodiment.

The foregoing formulations and administration methods are intended to be illustrative and not limiting. It will be appreciated that, using the teaching provided herein, other suitable formulations and modes of administration can be readily devised. Formulations of the present invention suitable for oral administration may be presented as discrete units such as capsules, cachets, tablets, lozenges comprising the peptide(s) in a flavoured base, usually sucrose and acacia and tragacanth; pastilles comprising the active ingredient(s) in an inert base such as gelatin and glycerin, or sucrose and acacia; and mouth washes comprising the active ingredient(s) in a suitable liquid carrier. Each formulation generally contains a predetermined amount of the active peptide(s); as a powder or granules; or a solution or suspension in an aqueous or non-aqueous liquid such as a syrup, an elixir, an emulsion or draught and the like.

A tablet may be made by compression or moulding, optionally with one or more accessory ingredients. Compressed tablets may be prepared by compressing in a suitable machine the active peptide(s) in a free-flowing form such as a powder or granules, optionally mixed with a binder, (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose), surface active or dispersing agent. Moulded tablets may be made by moulding in a suitable machine a mixture of the powdered peptide(s) moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile.

A syrup may be made by adding the active peptide(s) to a concentrated, aqueous solution of a sugar, for example, sucrose, to which may also be added any necessary ingredients. Such accessory ingredients) may include flavourings, an agent to retard crystallisation of the sugar or an agent to increase the solubility of any other ingredients, such as a polyhydric alcohol, for example, glycerol or sorbitol.

In addition to the aforementioned ingredients, the formulations of this invention may further include one or more accessory ingredient(s) selected from diluents, buffers, flavouring agents, binders, surface active agents, thickeners, lubricants, preservatives, (including antioxidants) and the like.

According to some embodiments of the invention, the therapeutically effective amount of the Hsp fragment or analog is a dosage in a range from about 0.02 mg/kg to about 10 mg/kg. Preferably, the dosage of the Hsp fragment or analog according to the present invention is in a range from about 0.025 mg/kg to about 5 mg/kg, more preferably, the dosage of the Hsp fragment or analog is in a range from about 0.025 mg/kg to about 1 mg/kg. It will be understood that the dosage may be an escalating dosage so that low dosage may be administered first, and subsequently higher dosages may be administered until an appropriate response is achieved. Also, the dosage of the composition can be administered to the subject in multiple administrations in the course of the treatment period in which a portion of the dosage is administered at each administration.

### Depot Systems

The parenteral route by intravenous (IV), intramuscular (IM), or subcutaneous (SC) injection is the most common and effective form of delivery for small as well as large molecular weight drugs. However, pain, discomfort and inconvenience due to needle sticks makes this mode of drug delivery the least preferred by patients. Therefore, any drug delivery technology that can at a minimum reduce the total number of injections is preferred. Such reductions in frequency of drug dosing in practice may be achieved through the use of injectable depot formulations that are capable of releasing drugs in a slow but predictable manner and consequently improve compliance. For most drugs, depending on the dose, it may be possible to reduce the injection frequency from daily to once or twice monthly or even longer (6 months). In addition to improving patient comfort, less frequent injections of drugs in the form of depot formulations smoothes out the plasma concentration-time profile by eliminating the hills and valleys. Such smoothing out of plasma profiles has the potential to not only boost the therapeutic benefit in most cases, but also to reduce any unwanted events, such as immunogenicity etc. often associated with large molecular weight drugs.

Microparticles, implants and gels are the most common forms of biodegradable polymeric devices used in practice for prolonging the release of drugs in the body. Microparticles are suspended in an aqueous media right before injection and one can load as much as 40% solids in suspensions. Implant/rod formulations are delivered to SC/IM tissue with the aid of special needles in the dry state without the need for an aqueous media. This feature of rods/implants allows for higher masses of formulation, as well as drug content to be delivered. Further, in the rods/implants, the initial burst problems are minimized due to much smaller area in implants compared to the microparticles. Besides biodegradable systems, there are non-biodegradable implants and infusion pumps that can be worn outside the body. Non-biodegradable implants require a doctor's visit not only for implanting the device into the SC/IM tissue but also to remove them after the drug release period.

Injectable compositions containing microparticle preparations are particularly susceptible to problems. Microparticle suspensions may contain as much as 40% solids as compared with 0.5-5% solids in other types of injectable suspensions. Further, microparticles used in injectable depot products, range in size up to about 250 μm (average, 60-100 μm), as compared with a particle size of less than sum recommended for IM or SC administration. The higher concentrations of solids, as well as the larger solid particle size require larger size of needle (around 18-21 gauge) for injection. Overall, despite the infrequent uses of larger and uncomfortable needles, patients still prefer less frequently administered dosage forms over everyday drug injections with a smaller needle.

Biodegradable polyesters of poly(lactic acid) (PLA) and copolymers of lactide and glycolide referred to as poly(lactide-co-glycolide) (PLGA) are the most common polymers used in biodegradable dosage forms. PLA is hydrophobic molecule and PLGA degrades faster than PLA because of the presence of more hydrophilic glycolide groups. These biocompatible polymers undergo random, non-enzymatic, hydrolytic cleavage of the ester linkages to form lactic acid and glycolic acid, which are normal metabolic compounds in the body. Resorbable sutures, clips and implants are the earliest applications of these polymers. Southern Research Institute developed the first synthetic, resorbable suture (Dexon®) in 1970. The first patent describing the use of PLGA polymers in a sustained release dosage form appeared in 1973 (U.S. Pat. No. 3,773,919).

Today, PLGA polymers are commercially available from multiple suppliers; Alkermes (Medisorb polymers), Absorbable Polymers International [formerly Birmingham Polymers (a Division of Durect)], Purac and Boehringer Ingelheim. Besides PLGA and PLA, natural cellulosic polymers such as starch, starch derivatives, dextran and non-PLGA synthetic polymers are also being explored as biodegradable polymers in such systems.

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the amended claims

### EXAMPLES

### Example 1

### Non-Obese Diabetic NOD Mice Model

Female non-obese diabetic (NOD) mice, which spontaneously develop autoimmune diabetes that mimics human T1D, are used to test the ability of the Hsp60 peptides and peptide analogs to induce regeneration of pancreatic beta cells. The NOD model is described, for example in Elias and Cohen (Lancet 1994, 343, 704-706). Female NOD mice raised under specific pathogen free (SPF) conditions develop insulitis around 4 weeks of age. Hyperglycemia begins at about 12-15 weeks and by 20-30 weeks almost all female NOD mice have developed serve diabetes and most die in the absence of insulin treatment. A revised NOD model is herein used, wherein treatment is began late in disease, after mice have lost most of their residual beta cells. In addition, the actual appearance of new beta cells, detected by increased C-peptide levels is measured in the revised protocol and not just preservation of initial C-peptide levels as in the original protocol.

Groups of 7 weeks old NOD/ShiLtJ mice (Jackson Number 001976, 10 mice per group) were used. Fasting blood glucose was monitored three times per week during all the treatment period, using methods known in the art. After about 12-20 weeks, when blood glucose reaches a level of 500 mg/CC the mice are treated with insulin in order to keep them alive and divided randomly between the following group treatments:
1. no treatment;
2. INTRALIPID® only, once a week;
3. 100 μg/ml DiaPep277 in INTRALIPID®, SC once a week;
4. 200 μg/ml DiaPep277 in INTRALIPID®, SC once a week;
5. 500 μg/ml DiaPep277 in INTRALIPID®, SC once a week;
6. 100 μg/ml DiaPep277 in PBS, IP three times a week.

Fasting glucose was measured Hemoglobin A1C and C-peptide levels were measured at the end of treatment, up to six months, depending on the conditions of the control and treated animals.

Histology was performed to detect and count beta cells in pancreatic islets and to observe islet infiltrates. Histology was performed by staining for insulin and detection of appearance of beta cells, as described for example in Lumelsky et al, Science 2001, 292, 1389-1394.

As demonstrated in Table 1, mice treated with DiaPep277 show increased number of beta islets and less lymphocyte infiltrates as compared to control mice treated with placebo.

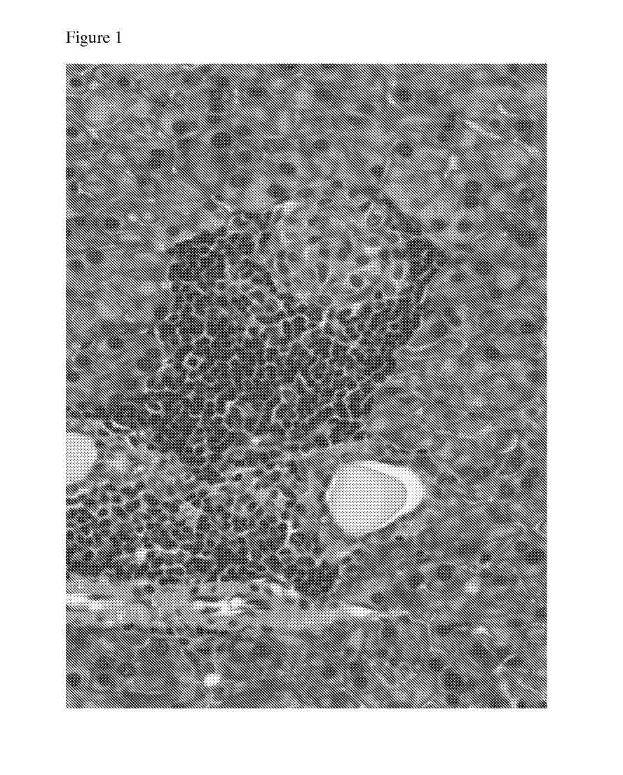

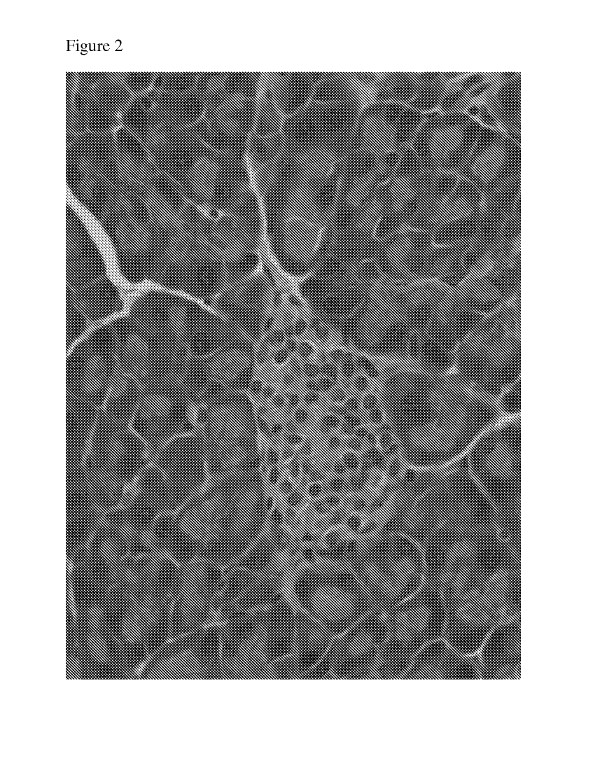

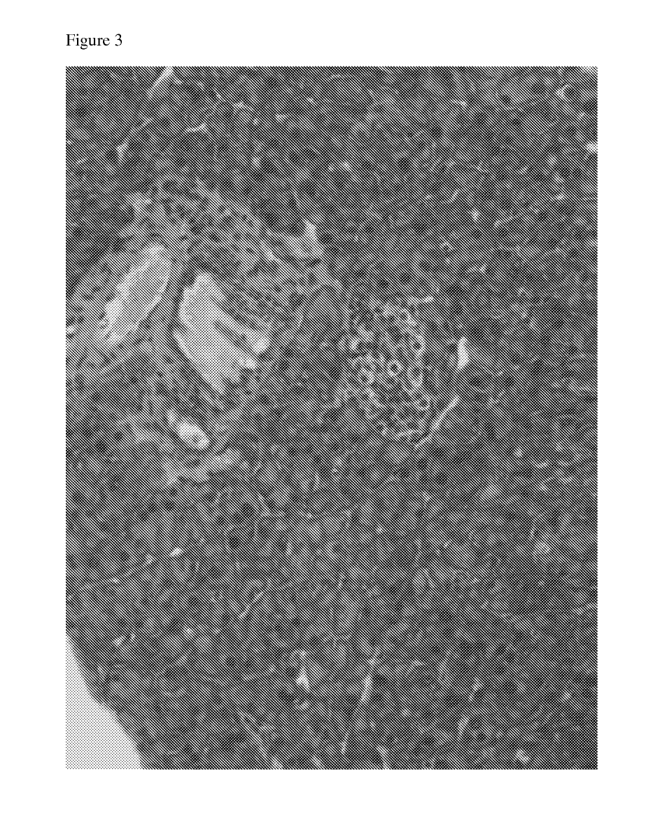

TABLE 1
Regeneration of islets in advanced T1D in NOD mice

| | | Injections/ | Islets per | Notes |
|---|---|---|---|---|
| Treatment | Route | week | whole section | Figure |
| Untreated | — | 0 | None | |
| INTRALIPID ® | SC | 1 | 2 | heavy infiltrate |
| | | | | FIG. 1 |
| DiaPep277 100 μg | IP | 3 | 2 | |
| in PBS | | | | |
| DiaPep277 100 μg | SC | 1 | 3 | no infiltrate |
| in INTRALIPID ® | | | | FIG. 2 |
| | | | | (slide 11) |
| DiaPep277 200 μg | SC | 1 | 1 | |
| in INTRALIPID ® | | | | |
| DiaPep277 500 μg | SC | 1 | 7 | no infiltrate |
| in INTRALIPID ® | | | | FIG. 3 (slide 7) |

The dose of 500 μg subcutaneous once a week gave the most islets per whole section while in both 100 and 500 μg doses (FIGS. 2 and 3), the islets were clear from lymphocyte infiltrates compared to the non-treated animals (FIG. 1).

### Example 2

### Animal Models for Beta-Cell Regeneration in Chemically-Induced Diabetes

The ability of DiaPep277 to induce or enhance beta cell regeneration is assessed in animal models in which diabetes is induce chemically and not via an autoimmune response (S. Lenzen, Diabetologia, 2008, 51:216-226).

In the high dose streptozotocin model (Arora et al., Global Journal of Pharmacology, 2009, 3: 81-84) a dose of 180 mg per kilo of streptozotocin injected IP destroys beta cells chemically and induces insulin-dependent diabetes within one week. This diabetes is not based on an autoimmune T cell effector reactive to beta cells, but is caused by direct chemical toxicity. This high dose streptozotcin model differs essentially from the repeated low dose model (40 mg/kg×5 described in Lukic et al., Developmental Immunology, 1998, Vol. 6, pp. 119-128) that induces an autoimmune disease.

In addition, the alloxan model of induced diabetes in mice is used in which chemical diabetes is induced with 70 mg per kilo of alloxan (Ingmar Lundquist and Claus Rerup, European Journal Of Pharmacology 1967, 2, 35-41).

The diabetic mice induced with alloxan or high dose streptozotocin are treated with DiaPep277 at doses of 200-500 micrograms per mouse (subcutaneous, IP, or oral; as with the NOD mice) beginning with the induction of the chemical diabetes and continuing 3 times a week to weekly or biweekly. And test for insulin levels, glucose levels, c-peptide, and glucose tolerance in Example 1.

While the present invention has been particularly described, persons skilled in the art will appreciate that many variations and modifications can be made. Therefore, the invention is not to be construed as restricted to the particularly described embodiments, rather the scope, spirit and concept of the invention will be more readily understood by reference to the claims which follow.

1.-33. (canceled)

34. A method of inducing regeneration of beta cell islets in a patient having T1D, the method consisting of administering a peptide derived from Hsp60 or a peptide analog thereof thereby increasing the beta cell mass.

35. The method of claim 34 wherein the peptide analog is 24-30 amino acids long and comprises a sequence corresponding to amino acid residues 437-460 of human Hsp60 having the sequence: Val-Leu-Gly-Gly-Gly-X₁-Ala-Leu-Leu-Arg-X₂-Ile-Pro-Ala-Leu-Asp-Ser-Leu-X₃-Pro-Ala-Asn-Glu-Asp (SEQ ID NO:1), wherein X₁ is a Cys or Val residue, X₂ is a Cys or Val residue, and X₃ is a Thr or Lys residue.

36. The method of claim 35 wherein the peptide analog it is a Val⁵, Val¹¹ analog of residues 437-460 of Hsp60, as set forth in SEQ ID NO:2:
(SEQ ID NO: 2)
1 6 11
Val-Leu-Gly-Gly-Gly-Val-Ala-Leu-Leu-Arg-Val-
Ile-Pro-Ala-Leu-Asp-Ser-Leu-Thr-Pro-
24
Ala-Asn-Glu-Asp, herein denoted DiaPep277.

37. The method of claim 34 wherein the Hsp60 peptide is selected from the group consisting of:
residues 31-50 of human Hsp60:
(SEQ ID NO: 3)
Lys-Phe-Gly-Ala-Asp-Ala-Arg-Ala-Leu-Met-Leu-
Gln-Gly-Val-Asp-Leu-Leu-Ala-Asp-Ala;
residues 136-155 of human Hsp60:
(SEQ ID NO: 4)
Asn-Pro-Val-Glu-Ile-Arg-Arg-Gly-Val-Met-Leu-
Ala-Val-Asp-Ala-Val-Ile-Ala-Glu-Leu;
residues 151-170 of human Hsp60:
(SEQ ID NO: 5)
Val-Ile-Ala-Glu-Leu-Lys-Lys-Gln-Ser-Lys-Pro-
Val-Thr-Thr-Pro-Glu-Glu-Ile-Ala-Gln;
residues 166-185 of human Hsp60:
(SEQ ID NO: 6)
Glu-Glu-Ile-Ala-Gln-Val-Ala-Thr-Ile-Ser-Ala-
Asn-Gly-Asp-Lys-Glu-Ile-Gly-Asn-Ile;
residues 195-214 of human Hsp60:
(SEQ ID NO: 7)
Arg-Lys-Gly-Val-Ile-Thr-Val-Lys-Asp-Gly-Lys-
Thr-Leu-Asn-Asp-Glu-Leu-Glu-Ile-Ile;
residues 255-274 of human Hsp60:
(SEQ ID NO: 8)
Gln-Ser-Ile-Val-Pro-Ala-Leu-Glu-Ile-Ala-Asn-
Ala-His-Arg-Lys-Pro-Leu-Val-Ile-Ile;
residues 286-305 of human Hsp60:
(SEQ ID NO: 9)
Leu-Val-Leu-Asn-Arg-Leu-Lys-Val-Gly-Leu-Gln-
Val-Val-Ala-Val-Lys-Ala-Pro-Gly-Phe;
residues 346-365 of human Hsp60:
(SEQ ID NO: 10)
Gly-Glu-Val-Ile-Val-Thr-Lys-Asp-Asp-Ala-Met-
Leu-Leu-Lys-Gly-Lys-Gly-Asp-Lys-Ala;
residues 421-440 of human Hsp60:
(SEQ ID NO: 11)
Val-Thr-Asp-Ala-Leu-Asn-Ala-Thr-Arg-Ala-Ala-
Val-Glu-Glu-Gly-Ile-Val-Leu-Gly-Gly;
residues 436-455 of human Hsp60:
(SEQ ID NO: 12)
Ile-Val-Leu-Gly-Gly-Gly-Cys-Ala-Leu-Leu-Arg-
Cys-Ile-Pro-Ala-Leu-Asp-Ser-Leu-Thr;
residues 466-485 of human Hsp60:
(SEQ ID NO: 13)
Glu-Ile-Ile-Lys-Arg-Thr-Leu-Lys-Ile-Pro-Ala-
Met-Thr-Ile-Ala-Lys-Asn-Ala-Gly-Val;
residues 511-530 of human Hsp60:
(SEQ ID NO: 14)
Val-Asn-Met-Val-Glu-Lys-Gly-Ile-Ile-Asp-Pro-
Thr-Lys-Val-Val-Arg-Thr-Ala-Leu-Leu;
residues 343-366 of human Hsp60:
(SEQ ID NO: 15)
Gly-Lys-Val-Gly-Glu-Val-Ile-Val-Thr-Lys-Asp-
Asp-Ala-Met.

38. The method according to claim 34 wherein administration is via a route selected from the group consisting of: intramuscular, intravenous, oral, intraperitoneal, subcutaneous, topical, intradermal or transdermal delivery.

39. The method according to claim 34 wherein the Hsp60 derived peptide or analog is administered in a pharmaceutical composition comprising at least one pharmaceutically acceptable diluent, excipient or carrier.

40. The method of claim 39 wherein the composition comprises the Hsp60 derived peptide or analog as the sole active ingredient.

41. The method according to claim 34 comprising administration of at least 2 mg of the Hsp60 derived peptide or peptide analog.

42. The method according to claim 34 comprising administration of 10 mg of the Hsp60 derived peptide or peptide analog.

43. The method according to claim 34 wherein the Hsp60 derived peptide or peptide analog is administered to a subject in need thereof 1-12 times per month.

44. The method according to claim 34 comprising administration of 2-10 mg of the Hsp60 derived peptide or peptide analog 1-4 times per month by a route selected from the group consisting of: subcutaneous injection, intra-peritoneal (IP) injection, intra-venous (IV) injection, intra-muscular (IM) injection and oral administration.

45. The method according claim 34 comprising oral administration of 50-500 mg Hsp60 peptide or analog 1-4 times per month.

46. A method of treating long established type 1 diabetes (T1D) comprising administering, at least once monthly, to a subject having a fasting C-peptide level of 0.2 nM or less or having a clinically established T1D for a period of one year or more, a composition comprising a dose of at least 2 mg of peptide derived from Hsp60 or a peptide analog thereof.

47. The method of claim 34 for treating T1D in a subpopulation of patients having no demonstrable functional pancreatic beta cells, as measured by plasma C-peptide levels, wherein a dose of at least 2 mg of the Hsp60 derived peptide or peptide analog is administered at least once monthly.

48. The method according to claim 46 wherein the Hsp60 derived peptide analog is

49. The method according to claim 47 wherein fasting plasma C-peptide level is 0.2 nM or less.

50. A long acting pharmaceutical composition comprising DiaPep277 (SEQ ID NO: 2) or a pharmaceutically acceptable salt thereof, specifically formulated for providing a therapeutically effective amount of the peptide over a period selected from 2-6 days, one week, two weeks or longer.

51. A method of inducing regeneration of beta cell islets in a patient having T1D comprising administering the long acting pharmaceutical composition of claim 50.

52. The method of claim 51 for treating subjects having a fasting C-peptide level of 0.2 nM or less or having a clinically established T1D for a period of a year or more.

53. The long acting pharmaceutical composition of claim 52 in depot form suitable for injection or implantation at a medically acceptable location in a subject in need thereof, and for a dosing schedule from once every 2 weeks to once monthly.


SEQUENCE LISTING (15 sequences)

SEQ ID NO: 1 - protein, 24 aa, Artificial Sequence
  Synthetic peptide
VLGGGXALLRXIPALDSLXPANED

SEQ ID NO: 2 - protein, 24 aa, Artificial Sequence
  Synthetic peptide
VLGGGVALLRVIPALDSLTPANED

SEQ ID NO: 3 - protein, 20 aa, Artificial Sequence
  Synthetic peptide
KFGADARALMLQGVDLLADA

SEQ ID NO: 4 - protein, 20 aa, Artificial Sequence
  Synthetic peptide
NPVEIRRGVMLAVDAVIAEL

SEQ ID NO: 5 - protein, 20 aa, Artificial Sequence
  Synthetic peptide
VIAELKKQSKPVTTPEEIAQ

SEQ ID NO: 6 - protein, 20 aa, Artificial Sequence
  Synthetic peptide
EEIAQVATISANGDKEIGNI

SEQ ID NO: 7 - protein, 20 aa, Artificial Sequence
  Synthetic peptide
RKGVITVKDGKTLNDELEII

SEQ ID NO: 8 - protein, 20 aa, Artificial Sequence
  Synthetic peptide
QSIVPALEIANAHRKPLVII

SEQ ID NO: 9 - protein, 20 aa, Artificial Sequence
  Synthetic peptide
LVLNRLKVGLQVVAVKAPGF

SEQ ID NO: 10 - protein, 20 aa, Artificial Sequence
  Synthetic peptide
GEVIVTKDDAMLLKGKGDKA

SEQ ID NO: 11 - protein, 20 aa, Artificial Sequence
  Synthetic peptide
VTDALNATRAAVEEGIVLGG

SEQ ID NO: 12 - protein, 20 aa, Artificial Sequence
  Synthetic peptide
IVLGGGCALLRCIPALDSLT

SEQ ID NO: 13 - protein, 20 aa, Artificial Sequence
  Synthetic peptide
EIIKRTLKIPAMTIAKNAGV

SEQ ID NO: 14 - protein, 20 aa, Artificial Sequence
  Synthetic peptide
VNMVEKGIIDPTKVVRTALL

SEQ ID NO: 15 - protein, 14 aa, Artificial Sequence
  Synthetic peptide
GKVGEVIVTKDDAM